(19) United States
(12) Patent Application Publication
(10) Pub. No.: US 2005/0142068 A1
(43) Pub. Date: Jun. 30, 2005

(54) Retinal toxicity screening methods

(75) Inventor: Maria E. Verdugo-Gazdik, Waterford, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 11/020,916

(22) Filed: Dec. 22, 2004

Related U.S. Application Data
(60) Provisional application No. 60/532,608, filed on Dec. 24, 2003.

(51) Int. Cl. A61K 49/00; A61K 38/12

(52) U.S. Cl. 424/9.6; 514/9

(57) ABSTRACT

The present invention relates to methods for characterizing a test agent using a fluorescently detectable αᵥβ₃ and αᵥβ₅ integrin specific agent and a retinal pigment epithelial cell. The invention further relates to kits having a fluorescently detectable αᵥβ₃ and αᵥβ₅ integrin specific agent and a retinal pigment epithelial cell.

### CROSS REFERENCE

This application claims the benefit of U.S. Provisional Patent Application No. 60/532,608, filed Dec. 24, 2003 and incorporated herein by reference in its entirety.

### FIELD OF THE INVENTION

The present invention relates to methods for characterizing a test agent using a fluorescently detectable αᵥβ₃ and αᵥβ₅ integrin specific agent and a retinal pigment epithelial cell.

### BACKGROUND OF THE INVENTION

The retina is a tightly compact, metabolically active, neural structure that is approximately 100 to 500 um in thickness, and occupies the innermost layer of the eye. Potts, A. M. (1996). Composed of distinct layers, the retina receives nourishment from the vascular choroid that lies just below the retinal pigment epithelium (RPE). Mayerson and Hall (1986).

A number of retinal conditions are known that can result in vision impairment and loss. The variety and causes of such conditions include diabetic retinopathy (diabetes), age-related macular degeneration (age), Stargardt's disease, retinitis pigmentosa (heredity), histoplasmosis (fungal infection) and retinopathy of prematurity (premature birth. Many of these conditions are characterized by the proliferation of new blood vessels (neovascularization) within the retinal tissue.

It is known that certain drugs can induce damage to the retina. For example retinopathy has been reported as a result of exposure to tamoxifen (Griffiths, M. F. (1987); Bentley, C. R., et al. (1992) and Pavlidis, N. A., et al. (1992)), cloroquine (Matsumura, M. M., et al. (1986); Fredman, P. G., et al. (1987); and Grant, W. M. and Schuman, J. S. (1993)), indomethacin (Burns, C. A. 1973), chlorpromazine and thioridazine (Siddall (1966)). The damage that is inducible by these drugs may involve neovascularization of retinal tissue.

Integrins are heterodimeric proteins that traverse the plasma membrane and provide specific points of attachment between cells, or between cells and extracellular matrix proteins. In addition to serving as cellular adhesives, integrins also play a role as receptors and signal transducers. Nelson, D. L. and Cox, M. M. (2000), p. 404.

Each integrin hetrodimer contains one α and β subunit. At least 16 different alpha subunits and at least 8 beta subunits have been reported. Aplin, A. E., et al. (1998).

The integrins, αᵥβ₃ and αᵥβ₅, are described to be strongly expressed on endothelial cells that have been activated (i.e., during neovascularization) as compared to non-activated endothelial cells. Friedlander, M., et al. (1995); Natali, P. G. et al. (1997).

The αᵥβ₃ and αᵥβ₅ integrins have been found to interact with certain proteins of the extracellular matrix that contain the triplet peptides Arg-Gly-Asp (RGD). DePasquale, J. A. (1998), Germer, M. et al. (1998) and International Patent Application Publication No.'s WO 97/06791 and WO 95/25543. αᵥβ₃ and αᵥβ₅ have been associated with angiogenesis. As such, RGD containing small peptides have been proposed as antagonists against vascular endothelial cell and tumor growth. Goligorsky, M. S. et al. (1998) and Sheu, J. R. et al. (1997).

It has been reported that the aspartic acid residue of RGD is highly susceptible to chemical degradation leading to a loss of biological activity, but that this degradation was prevented when the RGD-containing peptide was cyclized via disulfide linkage. Bogdanowich-Knipp, S. J. et al. (1999-A) and Bogdanowich-Knipp, S. J. et al. (1999-B).

It has been proposed that RGD peptides be used as markers for tumor imaging, for example, by labeling the peptides with the isotope, technetium-99m. Zi-Fen, S. et al. (2002).

International Patent Application Publication No.'s WO 01/77145 and WO 03/006491 disclose peptide-based compounds that bind αᵥ integrins and their use in the diagnosis of malignant diseases, such as, heart disease, endometriosis, inflammation-related diseases, rheumatoid arthritis and Kaposi's sarcoma.

International Publication Number WO 03/037172 discloses peptides and their derivatives and their use to inhibit angiogenesis and angiogenesis-related diseases such as cancer, arthritis, macular degeneration and diabetic retinopathy.

Several industries, including those that produce chemicals, cosmetics and food additives, as well as the pharmaceutical industry have a primary interest to ensure that the safety risk of their products is minimized. As such, there exists a need for new in vitro methods that provide a reliable and accurate assessment of potential chemical agent-induced retinal toxicity.

### SUMMARY OF THE INVENTION

The present invention relates, in part, to methods for characterizing a test agent comprising, treating a mammalian retinal pigment epithelial cell with a test agent, treating said cell with an integrin marker, exposing the cell to a light source having a wavelength that causes fluorescence of said integrin marker and detecting the fluorescence emitted by said integrin marker.

A further aspect of the invention provides methods for characterizing a test agent comprising, treating a first mammalian retinal pigment epithelial cell with a test agent, treating said first cell and a second mammalian retinal pigment epithelial cell with an integrin marker, exposing said first cell to a light source having a wavelength that causes fluorescence of said integrin marker and detecting the fluorescence emitted thereof and exposing said second cell to a light source having a wavelength that causes fluorescence of said integrin marker and detecting the fluorescence emitted thereof.

In a preferred embodiment, the methods further comprise characterizing said test agent according to a category selected from: an agent that causes an increase in the fluorescence emitted from a test mammalian retinal pigment epithelial cell as compared to a control mammalian retinal pigment epithelial cell; and an agent that does not cause an increase in the fluorescence emitted from a test mammalian retinal pigment epithelial cell as compared to a control mammalian retinal pigment epithelial cell, wherein said test cell has been treated with said test agent and said integrin marker and then exposed to a light source having a wavelength that causes fluorescence of said integrin marker; and wherein said control cell has been treated with said integrin marker and then exposed to a light source having a wavelength that causes fluorescence of said integrin marker. Preferably, said increased fluorescence is statistically significant. Alternatively, said increased fluorescence is preferably at least about two-fold.

Another aspect of the invention relates to kits comprising a retinal pigment epithelial derived cell, an integrin marker and packaging materials.

A preferred cell for use in the practice of the invention is a cell selected from an RPE-J cell and an ARPE-19 cell, or a cell derived thereof.

A preferred integrin marker for use in the practice of the invention is disulfide [Cys²⁻⁶] thioether cyclo [CH₂CO-Lys (fluorescein)-Cys²-Arg-Gly-Asp-Cys⁶-Phe-Cys]-(PEG)-NH₂.

In a more preferred embodiment of the invention, said cell is selected from an RPE-J cell and an ARPE-19 cell, or a cell derived thereof, and said integrin marker is disulfide [Cys²⁻⁶] thioether cyclo [CH₂CO-Lys (fluorescein)-Cys²-Arg-Gly-Asp-Cys⁶-Phe-Cys]-(PEG)-NH2.

### BRIEF DESCRIPTION OF THE DRAWINGS

FIG. 1 depicts the viability of RPE-J cells following treatment with varying concentrations of tamoxifen as a percentage of control untreated cells using the WST-1 colorimetric assay. Each data point also shows standard error of measurement (SEM) limits.

FIGS. 2-5 are confocal photomicrographs of rat retinal pigment epithelial cells (RPE-J) treated with the integrin peptide marker, disulfide [Cys²⁻⁶] thioether cyclo [CH₂CO-Lys (fluorescein)-Cys²-Arg-Gly-Asp-Cys⁶-Phe-Cys]-(PEG)-NH2, the DNA stain TOTO®-3 iodide and varying concentrations of the retinal toxicant, tamoxifen. For each figure, the photomicrograph on the left is made using two excitation wavelengths—488 nm (argon laser) for the integrin marker peptide and 633 nm (HeNe laser) for TOTO®-3 iodide. The integrin peptide marker fluoresces at 520 nm and TOTO®-3 iodide fluoresces at 660 nm. The photomicrograph on the right is of the identical cells as those in the left micrograph, but under 488 nm excitation alone.

FIG. 2 depicts RPE-J cells that were treated with no tamoxifen. As illustrated by the photomicrograph on the right, no integrin peptide marker is detectable in such cells.

FIG. 3 depicts RPE-J cells that were treated with 1 μM tamoxifen. As illustrated by the photomicrograph on the right, the integrin peptide marker is clearly visible.

FIG. 4 depicts RPE-J cells that were treated with 25 μM tamoxifen. As illustrated by the photomicrograph on the right, the integrin peptide marker is highly visible.

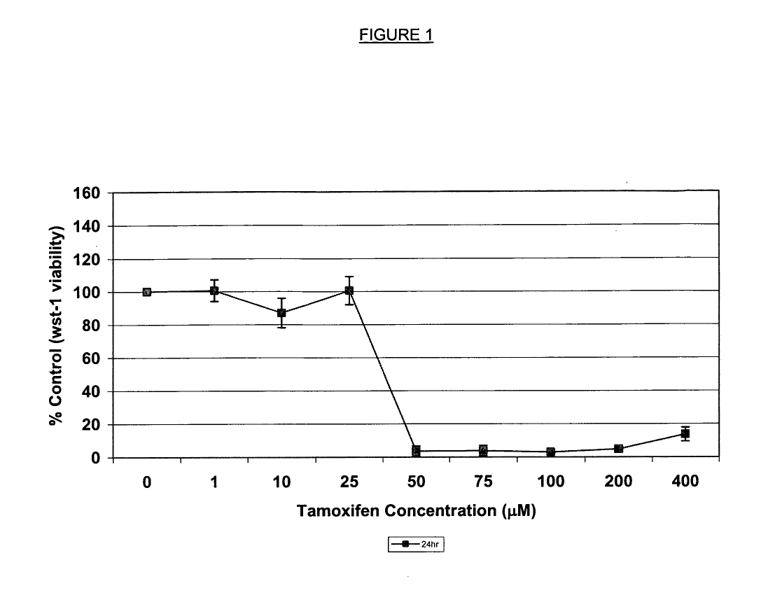

### DETAILED DESCRIPTION OF THE INVENTION

The terms used herein have their usual meaning in the art. However, to even further clarify the present invention and for convenience, the meaning of certain terms and phrases employed in the specification, including the examples and appendant claims are provided below.

“Integrin marker” means a fluorescently detectable αᵥβ₃ and αᵥβ₅ integrin-specific agent.

“αᵥβ₃ and αᵥβ₅ integrin specific agent” means a chemical agent that specifically binds either or both of the αᵥβ₃ or αᵥβ₅ integrin subunits.

The terms “specific binding” and “specifically binding” when referring to a protein, refer to a binding reaction that is determinative of the presence of the protein in a heterogeneous population of proteins and other biological components. Thus, for example, the specific binding of an integrin marker to an αᵥβ₃ and/or αᵥβ₅ integrin protein is sufficiently higher than the binding that occurs in the background (i.e., to non-αᵥβ₃ or αᵥβ₅ integrins) as to enable detection of the presence of αᵥβ₃ and/or αᵥβ₅ integrins from among the background. Preferably, the binding affinity for specific binding is at least twice that which occurs in the background.

The abbreviations used herein have their usual meaning in the art. However, to even further clarify the present invention, for convenience, the meaning of certain abbreviations are provided as follows: “° C.” means degrees centigrade; “DMF” means dimethylformamide; “ATCC” means the American Type Culture Collection located in Manassas, Va. (website at www.atcc.org); “CO₂” means carbon dioxide; “DMEM” means Dulbecco's modified Eagle's medium; “DNA” means deoxyribonucleic acid; “EDTA” means ethylenediamine tetra-acetic acid; “g” means gram; “kg” means kilogram; “mg” means milligram; “mL” means milliliter; “mM” means millimolar; “μl” means microliter; “μM” means microimolar; “ng” means nanogram; “nm” means nanometer; “nM” means nanomollar; “RNA” means ribonucleic acid; and “RPM” means revolutions per minute.

In one embodiment of the invention, an agent is characterized for its retinal toxicity potential by treating a mammalian retinal pigment epithelial cell with a test agent and a fluorescently detectable integrin marker and measuring the effect of the test agent on the level of fluorescence of the cell.

Any suitable mammalian retinal pigment epithelial cell may be used in the methods of this invention, including epithelial cells obtained directly from mammalian retinal tissue and cells derived from mammalian retinal pigment epithelial cell lines. Those with skill in the art will appreciate that, when predicting retinal toxicity risk in a particular mammalian species, for example, human, by the methods of this invention it may be preferable to use epithelial cells derived from that same species.

Primary retinal pigment epithelial cells may be harvested from retinal tissue according to methods known to those with skill in the art, for example, as described in Verdugo, M. E, et al. (2001) and Verdugo, M. E and Ray, J. (1997). Mammalian retinal pigment epithelial cell lines may be prepared by methods known to those with skill in the art, based upon the present disclosure. For example, primary retinal pigment epithelial cells harvested by known methods may be transformed with oncogenes or viral proteins. Nabi, I. et al. (1993) describes such a method by infecting rat primary retinal pigment epithelial cells with a temperature-sensitive SV40 virus. Alternatively, retinal pigment epithelial cells have been observed to arise spontaneously in primary cell cultures (e.g., see Dunn K. C. et al. (1996); and McLaren et al. (1993)).

In a preferred embodiment, the retinal pigment epithelial cells are derived from the rat retinal pigment epithelial cell line, RPE-J, or the human retinal pigment epithelial cell line, ARPE-19. Both the RPE-J and ARPE-19 cell lines are readily available, for example, from the ATCC (catalogue numbers CRL-2240 and CRL-2302, respectively).

Retinal pigment epithelial cell lines may be maintained according to methods known to those with skill in the art, for example, as generally described in Bonifacino et al. (1998). Exemplary culturing methods for RPE-J and ARPE-19 cells lines are disclosed in Nabi et al. (1996) and Dunn, et al. (1996) respectively. In a preferred method for RPE-J cells, the cells are cultured in high glucose Dulbecco's modified Eagle's medium (DMEM), containing 4% fetal calf serum (FCS) at about 33° C. When the cells reach a suitable level of confluence (e.g., 80%), they are dissociated using a 0.25% trypsin solution and replated at a ratio of approximately 1:4.

As those with skill in the art will appreciate based upon the present disclosure, any suitable integrin marker may be used in the practice of this invention. Generally, such integrin markers will have the αᵥβ₃ and αᵥβ₅ integrin specific tripeptide sequence, arginine-glycine-aspartic acid (RGD), as part of their chemical structure. Preferably, such RGD-containing peptides are cyclized via disulfide linkage in order to prevent chemical degradation of the aspartic acid residue. Bogdanowich-Knipp, S. J. et al. (1999-A) and Bogdanowich-Knipp, S. J. et al. (1999-B).

European Patent Application EP 578083 describes a series of mono-cyclic RGD containing peptides. Multi-bridged cyclic RGD peptides are described in International Patent Application Publication No.'s WO 98/54347 and WO 95/14714. Additional cyclic RGD peptides are described in Bogdanowich-Knipp, S. J. et al. (1999-A) and Bogdanowich-Knipp, S. J. et al. (1999-B).

As those with skill in the art will appreciate based upon the present disclosure, any suitable fluorescent label may be used to label the integrin marker. For example, fluorescent labels may include any fluorescein or rhodamine.

Exemplary integrin markers include RGD peptides disclosed in International Patent Application publications WO 03/006491 and WO 01/77145. A preferred marker is the fluorescein bound di-cyclic RGD peptide compound, disulfide [Cys²⁻⁶] thioether cyclo [CH₂CO-Lys (fluorescein)-Cys²-Arg-Gly-Asp-Cys⁶-Phe-Cys]-(PEG)-NH2, having the structure of Formula I:

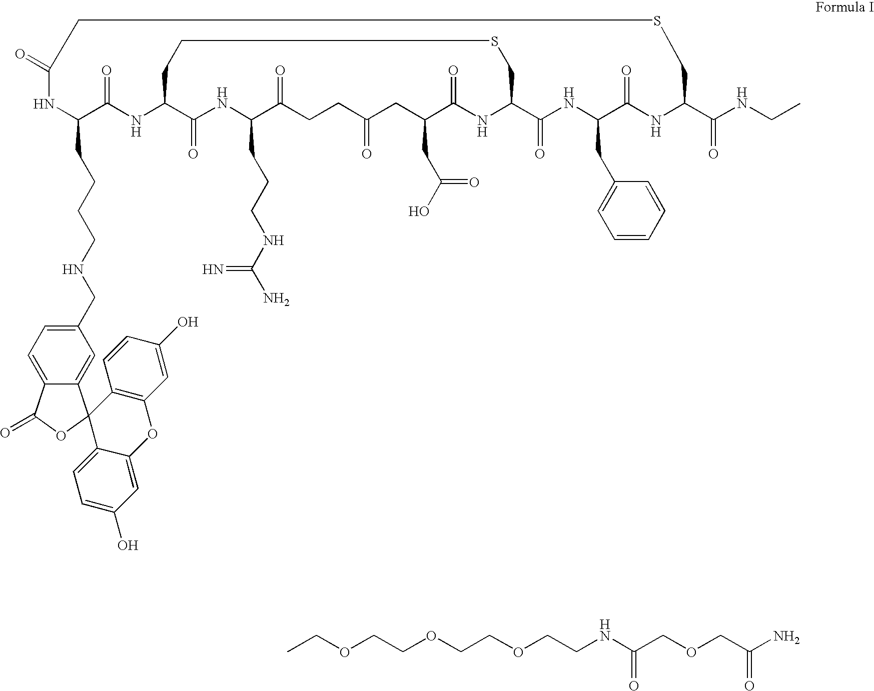

The integrin marker of Formula I may be prepared, for example, by first preparing the RGD peptide compound, disulfide [Cys²⁻⁶] thioether cyclo [CH₂CO-Lys-Cys²-Arg-Gly-Asp-Cys⁶-Phe-Cys]-(PEG)-NH2 according to methods described in WO 03/006491 and then reacting the RGD compound with NHS-fluorescein according to the procedure more fully described in the Examples.

In the practice of the methods of the invention, the treatment of cells with a test agent may be employed according to methods known by those with skill in the art based upon the present disclosure. Those with skill in the art will also appreciate that the method used will depend upon many variables, including the types of cells used, characteristics of the fluorescently detectable αᵥβ₃ integrin specific agent and characteristics of the test agent used.

In one embodiment, pigment epithelial cells are plated, preferably, at about 50,000 cells per milliliter onto multiwell plates and allowed to reach about 80% confluence. The cells are then treated with the test agent and the integrin marker together for about one hour.

As will be appreciated by those with skill in the art based upon the present disclosure, the amount of test agent in the practice of the invention used will depend upon many factors, including the types of cells used and characteristics of the test agent. Preferably, the amount of test agent used is less than that which has a substantially effect on the viability of the cells as a result of general toxicity. Conversely, sufficient test agent should be used that would enable a determination of whether the agent is likely to cause retinal toxicity based upon increased αᵥβ₃ and αᵥβ₅ integrins expression. Hence, the amount of test agent that is used should be the maximum amount which still does not cause a substantial decrease in viability.

Cell viability assays are well known to those with skill in the art. An exemplary cell viability method is the colorimetric WST-1 cytotoxicity assay described in the Roche Molecular Biochemicals manual entitled “Apoptosis and Cell” (internet address biochem.boehringer-mannheim.com/PROD INF/MANUALS/cell man/cell). Reagents for the WST-1 assay are available from Roche Diagnostics Corp., Indianapolis, Ind. (catalogue no. 1644807).

Likewise, the amount of integrin marker used should be less than an amount that would cause a decrease in viability. A cell viability assay such as the WST-1 assay described above may also be used to determine the upper limit of the integrin marker in the same manner used to determine the upper limit for the test agent. Moreover, the amount of integrin marker should be sufficiently low so as to minimize the effects of background fluorescence, but sufficiently high to enable detection of those cells which elicit a positive response due to increased expression of αᵥβ₃ and αᵥβ₅ integrins. The lower limit may be determined by the use of a positive control agent that is known to elecit increased expression of αᵥβ₃ and αᵥβ₅ integrins.

Following treatment of the cells with the test agent and integrin marker, the cells are preferably fixed with a fixative agent, e.g. paraformaldehyde or glutaraldehyde. Methods for fixing cells are known to those with skill in the art based upon the present disclosure. For example, Bacallo et al. (1990) and Bacallo and Garfinkel (1994) discuss in detail aspects about cell fixation.

At the time of fixation, the cells may be treated with a counter-stain, such as a DNA counter-stain. A counter-stain would be such that it fluoresces at a different wavelength from that of the integrin marker and enables identification of individual cells. Exemplary DNA counter-stains are TOTO®-3 iodide, SYBR Green I or propidium iodide (all available from Molecular Probes, Eugene, Oreg.). When using a DNA counter-stain, cells should also be treated with RNase to limit staining to the DNA.

Treated and stained cells are visualized by methods well known to those with skill in the art based upon the present disclosure. In a preferred embodiment, cells are visualized using confocal microscopy by methods known to those with skill in the art, including those described in Cheng et al. (1994), Gogswell and Carlsson (1994), Matsumoto (1993), Pawley (1990) and Stevens et al. (1994).

As those with skill in the art will appreciate based upon the present disclosure, the particular integrin marker used will have a characteristic light wavelength for excitation and fluorescence. For example, when the fluorescein bound di-cyclic RGD peptide compound, disulfide [Cys²⁻⁶] thioether cyclo [CH₂CO-Lys (fluorescein)-Cys²-Arg-Gly-Asp-Cys⁶-Phe-Cys]-(PEG)-NH2, is excited by light at about 488 nm, it emits light at about 520-560 nm with a peak emission at about 530 nm. For other integrin markers, the optimal excitation and emission wavelengths can be readily determined by methods well known to those with skill in the art, for example, using a fluorometer such as the TD-700 Laboratory Fluorometer (Turner BioSystems, Inc., Sunnyvale, Calif.).

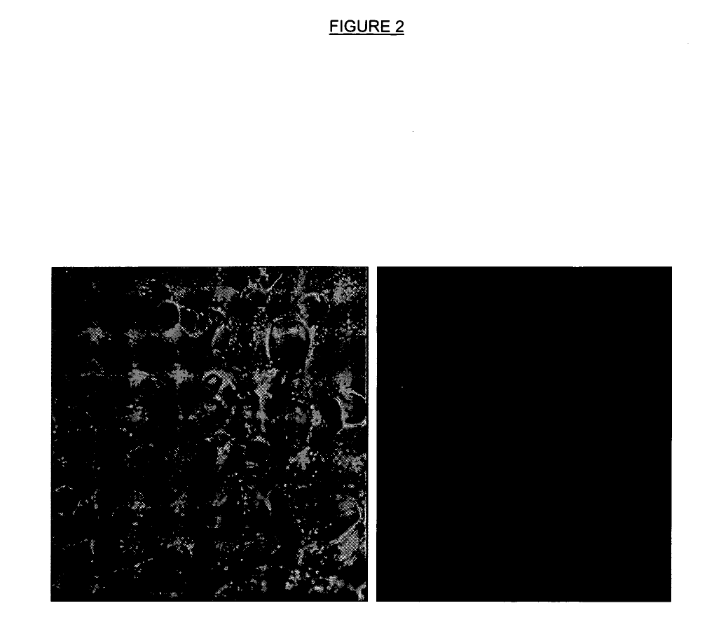

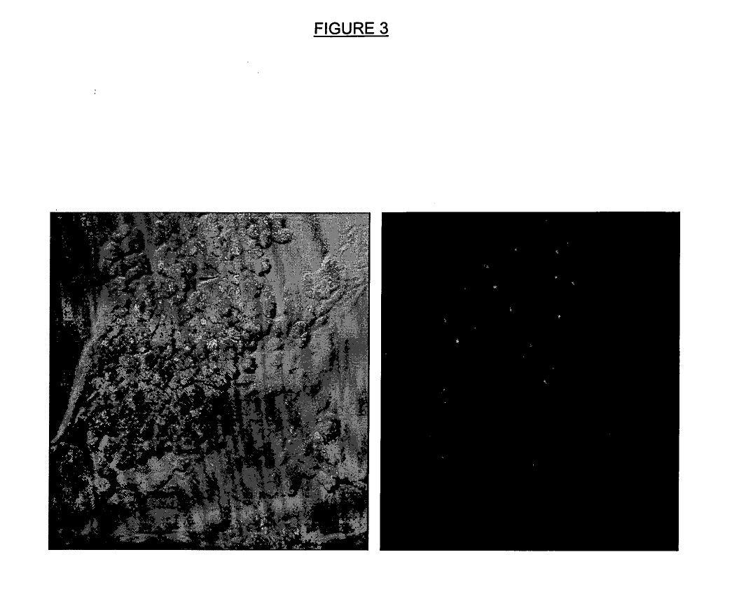

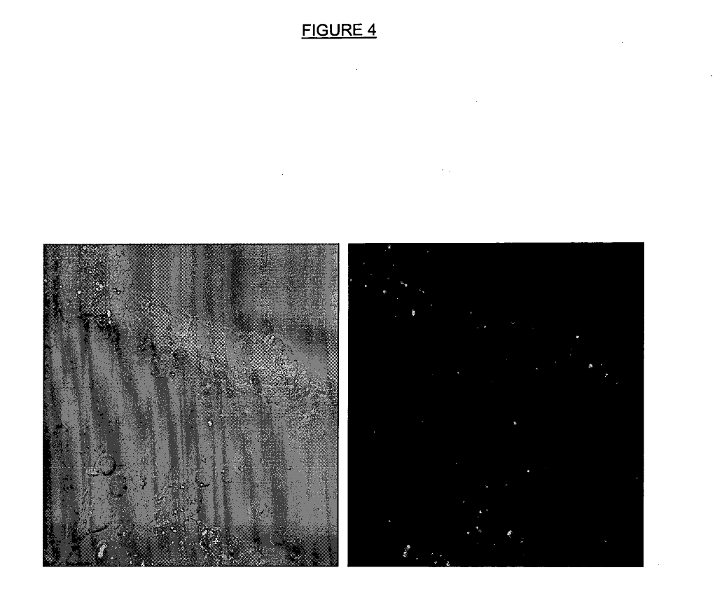

Generally, cells that exhibit fluorescence of the integrin marker specific agent will be clearly visible as compared to control cells (see FIGS. 2-5). Such cells will indicate a test agent that is a potential retinal toxicant.

As will be appreciated by those with skill in the art based upon the present disclosure, the methods of this invention may be adapted for automated testing of agents for likelihood of retinal toxicity. Such methods may, for example, involve automation of the detection of the integrin marker, such as through the use of laser scanning cytometry (LSC) method and instrument available from CompuCyte Corporation, Cambridge, Mass., USA. For such methods, any statistically significant increase in the level of fluorescence of the integrin marker in treated cells as compared to untreated cells will be indicative of a test agent having potential retinal toxicity properties. The determination of statistical significance is well known to those with skill in the art or will be apparent based upon the present disclosure. Preferably, the results will yield a p-value that is no more than 0.05, more preferably no more than 0.01 (Brownlee (1960)). Alternatively, the increase in fluorescence of the integrin marker in treated cells is at least about 2-fold over the control.

The above-described methods are for illustrative purposes only. Those with skill in the art will appreciate based upon the present disclosure that a variety of formats may be utilized in the practice of this invention. Variations may be made based upon the types of cells, integrin markers and test agents used, methods of treating and culturing cells and methods of detection of fluorescence.

The disclosures of all patents, applications, publications and documents, including brochures and technical bulletins, cited herein, are hereby expressly incorporated by reference in their entirety. It is believed that one skilled in the art can, based on the present description, including the examples, drawings, and attendant claims, utilize the present invention to its fullest extent.

The following Examples are to be construed as merely illustrative of the practice of the invention and not limitative of the remainder of the disclosure in any manner whatsoever.

### EXAMPLES

### Example 1

### Preparation of Disulfide [Cys²⁻⁶] Thioether Cyclo [CH₂CO-Lys (fluorescein)-Cys²-Arg-Gly-Asp-Cys⁶-Phe-Cys]-(PEG)-NH2

Thirty milligrams of disulfide [Cys²⁻⁶] thioether cyclo [CH₂CO-Lys-Cys²-Arg-Gly-Asp-Cys⁶-Phe-Cys]-(PEG)-NH₂, prepared by method described in Example 2 of International Patent Application Publication No. WO 03/006491, is dissolved in DMF (3 mL) together with NHS-fluorescein (16.2 mg) and N-methylmorpholine (4 μl). The mixture is protected from light and stirred overnight. The mixture is then purified by HPLC (Vydac 218TP1022 C18 column) using 20-30% B, where A=H₂O/0.1% TFA and B=CH₃CN/0.1 TFA, over 40 minutes at a flow rate of 10 mL/minute. The resulting fraction is lyophilized to yield 21.6 mg of the title compound.

Analytical GPLC: gradient, 10-40% B over 10 minutes where A=H₂O/0.1% TFA and B=CH₃CN/0.1% TFA; column, Phenomenex Luna 3μ C18 (2) 50×4.6 mm; flow, 2 mL/minute; detection, UV 214 nm; product retention time 7.0 minutes. Mass spectrometry: expected, M⁺H at 1616.5, found at 1616.3.

Example 1 illustrates a method for preparing the integrin marker, disulfide [Cys²⁻⁶] thioether cyclo [CH₂CO-Lys-Cys²-Arg-Gly-Asp-Cys⁶-Phe-Cys]-(PEG)-NH₂, for use in the methods of the invention.

### Example 2

### Assay to Identify Retinal Toxic Agents

RPE-J cells were plate cultured in high glucose DMEM medium (cat. no. 10313021, Invitrogen, Carlsbad, Calif.) containing 4% fetal calf serum (cat. no. 12319018, Invitrogen) at 33° C. under 5-10% CO₂ atmosphere. When confluence of about 80% was reached (after about seven days), the medium was removed from the culture dish and the cells on the bottoms were washed with 0.25% trypsin-EDTA (cat. no. 25200056, Invitrogen) for three and one-half to five minutes. Following trypsinization the cells were centrifuged at 500-600 RPM for five minutes and resuspended in high glucose DMEM medium containing 4% fetal calf serum. The cells were plated in a four-chamber slide (1.7 cm² per chamber) at 50,000 cells per chamber. When confluence of about 80% was reached, the cells were gently wash the cells in warm phosphate buffered saline. High glucose DMEM (500 μl) containing 0.01% bovine serum albumin, 50 nM of the integrin marker peptide, disulfide [Cys²⁻⁶] thioether cyclo [CH₂CO-Lys (fluorescein)-Cys²-Arg-Gly-Asp-Cys⁶-Phe-Cys]-(PEG)-NH2, prepared according to Example 1 and either 0, 1, 12.5 or 25 μM of tamoxifen was added to each chamber. After one hour of incubation at 37° C., the media was removed and the cells were fixed for 30 minutes in a solution of phosphate buffered saline (500 μl per chamber) containing 4% paraformaldehyde (cat. no. 15713, Electron Microscopy Sciences, Fort Washington, Pa.) and then incubated for one hour with TOTO®-3 iodide (Molecular Probes, Eugene, Oreg.) and RNase (Sigma Aldrich Co., St. Louis, Mo.) at a concentration of one mg/mL. The resulting cells where visualized by confocal imaging using Leica SP Laser Scanning Microscope (Leica Microsystems Inc., Bannockburn, Ill.) at 40× objective using oil (zoom 1.15). The wavelength used for excitation was 488 nm (argon laser) for the integrin marker peptide and 633 nm (HeNe laser) for TOTO®-3 iodide. Fluorescence was detected at 520 nm for the integrin marker peptide and at 660 nm for TOTO®-3 iodide.

Example 2 illustrates one embodiment of the invention wherein a test agent is characterized for its retinal toxicity potential as measured by the level of fluorescence following treatment with an integrin marker.

### LITERATURE

Aplin, A. E., et al. (1998), “Signal transduction and signal modulation by cell adhesion receptors: the role of integrins, cadherins, immunoglobulin-cell adhesion molecules, and selectins,” Pharmacological Rev., 50(2), 197-263;
Bacallo, R., et al. (1990) “Guiding principles of specimen preservation for confocal fluorescence microscopy,” Handbook of Biological Confocal Microscopy (J. Pawley, ed.) Plenum, New York, 197-205;
Bacallo, R., and Garfinkel, A. (1994), Three-Dimensional Confocal Microscopy: Volume Investigation of Biological Systems, (Stevens, J. K. et al., eds.), Academic Press, London, 172-4;
Bentley, C. R., et al. (1992), “Tamoxifen retinopathy: a rare but serious complication,” Brit. Med. J. 304, 495-6;
Bogdanowich-Knipp, S. J. et al. (1999-A), “The effect of conformation on the solution stability of linear vs. cyclic RGD peptides,” J. Pept. Res. 53(5), 523-9;
Bogdanowich-Knipp, S. J. et al. (1999-B), “Solution stability of linear vs. cyclic RGD peptides,” J. Pept. Res. 53(5), 530-41;
Bonfacino, J. S., et al. (eds.) (1998), Current Protocols in Cell Biology, John Wiley & Sons, Hoboken, N.J.;
Burns, C. A. (1973), “Indomethacin induced ocular toxicity,” Am. J. Ophthalmol., 76, 312-313;
Cheng, P. C. et al. eds. (1994), Multidimensional Microscopy, Springer Verlag, New York;
Cogswell, C. J. and Carlsson, K. (1994) Three-dimensional microscopy: image acquisition and processing, SPIE. Bellingham, Wash., USA;
Davis, A. et al. (1995), “A human retinal pigment epithelial cell line that retains epithelial characteristics after prolonged culture,” Invest. Opthamol. Vis. Sci., 36, 955-64;
DePasquale, J. A. (1998), “Cell matrix adhesions and localization of the vitronectin receptor in MCF-7 human mammary carcinoma cells,” Histochem. Cell Biol., 110(5), 485-94;
Dunn K. C. et al. (1996), “ARPE-19, A human retinal pigment epithelial cell line with differentiated properties,” Exp. Eye Res., 62, 155-169;
Fredman, P. G., et al. (1987), “Effect of chloroquine on the activity of some lysosomal enzymes involved in ganglioside degradation,” Biochim. Biophys. Acta, 917, 1-8;
Friedlander, M., et al. (1995), “Definition of two aniogenic pathways by distinct αᵥ integrins,” Science 270, 1500-2;
Germer, M. et al. (1998), “Kinetic analysis of integrin-dependent cell adhesion on vitronectin—the inhibitory potential of plasminogen activator inhibitor-1 and RGD peptides,” Euro. J. Biochem. 253(3), 669-74;
Goligorsky, M. S. et al. (1998), “Therapeutic effect of arginine-glycine-aspartic acid peptides in acute renal injury,” Clin. Exp. Pharmacol. Physiol. 25(3-4), 276-9;
Grant, W. M. and Schuman, J. S. (1993), Toxicology of the Eye, Charles C. Thomas, Springfield, Ill., 371-382;
Griffiths, M. F. (1987), “Tamoxifen retinopaty at low dosage,” Amer. J. Opththalmol., 104, 185-6;
Hendrix, M. J. et al. (2000), “Molecular biology of breast cancer metastasis. Molecular expression of vascular markers by aggressive breast cancer cells,” Breast Cancer Res. 2(6), 417-22;
Matsumoto, B., ed. (1993), Cell biological applications of confocal microscopy, Academic Press. San Diego, Calif.;
Matsumura, M. M., et al. (1986), “Experimental chloroquine retinopathy,” Ophthalmic Res., 18, 172-9;
Mayerson, P. L. and Hall, M. O. (1986), “Rat retinal pigment epithelial cells show specificity of phagocytosis in vitro,” J. Cell Biol., 103, 299-308
McLaren, M. et al. (1993), “Spontaneously arising immortal cell line of rat retinal pigmented epithelial cells,” Exp. Cell Res., 213, 85-92;
Nabi, I. et al. (1993), “Immortalization of polarized rat retinal pigment epithelium,” J. Cell Sci., 104, 37-49;
Natali, P. G. et al. (1997), “Clinical significance of alpha(v)-beta3 integrin and intercellular adhesion molecule-1 expression in cutaneous malignant melanoma lesions,” Cancer Res. 57(8), 1554-60;
Nelson, D. L. and Cox, M. M. (2000), Lehninger Principles of Biochemistry, 3^{rd} Ed., Worth Publishers, New York, N.Y.;
Pawley, J. B., ed. (1990), Handbook of Biological Confocal Microscopy, Plenum, New York;
Pavlidis, N. A., et al. (1992), “Clear evidence that long-term, low-dose tamoxifen treatment can induce ocular toxicity. A perspective study of 63 patients,” Cancer 69, 2961-4;
Potts, A. M. (1996), “Toxic Responses of the Eye,” Casarett & Doull's Toxicology: The basic Science of Poisons, Klaassen C D, Eds. New York, McGraw-Hill, 583-615;
Sheu, J. R. et al. (1997), “Inhibition of angiogenesis in vitro and in vivo: comparison of the relative activities of triflavin, an Arg-Gly-Asp-containing peptide and anti-alpha(v)beta3 integrin monoclonal antibody,” Biochim. Biophys. Acta. 1336(3), 445-54;
Siddall, J. R (1966), “Ocular toxic changes associated with chlorpromazine and thioridazine,” Can. J. Ophthalmol., 1, 190-8;
Stevens, J. K. et al., eds. (1994), Three-Dimensional Confocal Microscopy: Volume Investigation of Biological Systems, Academic Press, London;
Verdugo, M. E, et al. (2001), “Adenoviral vector-mediated beta-glucuronidase cDNA transfer to treat MPS VII RPE in vitro,” Curr Eye Res. 23(5), 357-67;
Verdugo, M. E and Ray, J. (1997), “Age-related increase in activity of specific lysosomal enzymes in the human retinal pigment epithelium,” Exp Eye Res. 65(2), 231-40; and
Zi-Fen, S. et al. (2002), “In vitro and in vivo evaluation of a technetium-99m-labelled cyclic RGD peptide as a specific marker of alpha(v)beta3 integrin for tumor imaging,” Bioconjugate Chem. 13, 561-70.

1. A method for characterizing a test agent, comprising, treating a mammalian retinal pigment epithelial cell with a test agent, treating said cell with an integrin marker, exposing the cell to a light source having a wavelength that causes fluorescence of said integrin marker and detecting the fluorescence emitted by said integrin marker.

2. A method for characterizing a test agent, comprising, treating a first mammalian retinal pigment epithelial cell with a test agent, treating said first cell and a second mammalian retinal pigment epithelial cell that has not been treated with said test agent with an integrin marker, exposing said first cell to a light source having a wavelength that causes fluorescence of said integrin marker and detecting the fluorescence emitted thereof and exposing said second cell to a light source having a wavelength that causes fluorescence of said integrin marker and detecting the fluorescence emitted thereof.

3. A method of claim 2, further comprising, characterizing said test agent according to a category selected from:
an agent that causes an increase in the fluorescence emitted from a test cell as compared to a control cell; and
an agent that does not cause an increase in the fluorescence emitted from a test cell as compared to a control cell,
wherein said test cell is defined as a mammalian retinal pigment epithelial cell that has been treated with said test agent and said integrin marker and then exposed to a light source having a wavelength that causes fluorescence of said integrin marker, and
wherein said control cell is defined as a mammalian retinal pigment epithelial cell that has been treated with said integrin marker, but not with said test agent, and then exposed to a light source having a wavelength that causes fluorescence of said integrin marker.

4. A method according to claim 3, wherein said increased fluorescence is statistically significant.

5. A method according to claim 3, wherein said increased fluorescence is at least two-fold.

6. A method according to any one of claims 1-5, wherein said cell is selected from an RPE-J cell and an ARPE-19 cell, or a cell derived thereof.

7. A method according to any one of claims 1-5, wherein said integrin marker is disulfide [Cys²⁻⁶] thioether cyclo [CH₂CO-Lys (fluorescein)-Cys²-Arg-Gly-Asp-Cys⁶-Phe-Cys]-(PEG)-NH2.

8. A method according to any one of claims 1-5, wherein said cell is selected from an RPE-J cell and an ARPE-19 cell, or a cell derived thereof, and said integrin marker is disulfide [Cys²⁻⁶] thioether cyclo [CH₂CO-Lys (fluorescein)-Cys²-Arg-Gly-Asp-Cys⁶-Phe-Cys]-(PEG)-NH₂.

9. A kit comprising a retinal pigment epithelial derived cell, an integrin marker and packaging materials.
